(19) United States
(12) Patent Application Publication
(10) Pub. No.: US 2014/0155887 A1
(43) Pub. Date: Jun. 5, 2014

(54) ABLATION COMPASS

(71) Applicant: Luca Neri, Baltimore, MD (US)

(72) Inventor: Luca Neri, Baltimore, MD (US)

(73) Assignee: THE JOHNS HOPKINS UNIVERSITY, Baltimore, MD (US)

(21) Appl. No.: 14/126,467

(22) PCT Filed: Jun. 6, 2012
(86) PCT No.: PCT/US12/41042

§ 371 Date: Feb. 23, 2014

Related U.S. Application Data
(60) Provisional application No. 61/497,332, filed on Jun. 15, 2011.

(51) Int. Cl. A61B 18/14

(52) U.S. Cl. CPC A61B 18/1492
USPC 606/41

(57) ABSTRACT

An embodiment in accordance with the present invention provides an ablation catheter includes a catheter body and an ablation electrode attached to the distal end of the catheter body. A pivot needle is arranged to extend from said catheter body while in use. The pivot needle provides support to assist with directing said ablation electrode along a selected path in contact with tissue during ablation. The pivot needle includes a gripping structure at a distal tip to facilitate attachment to tissue, and can take the form of a barb structure. The barb structure is formed from a soft material to facilitate removal from tissue after being attached. The pivot needle further includes a depth guard to help prevent the insertion of said pivot needle into tissue beyond a predetermined depth, and said pivot needle is extendable from and retractable to said catheter body.

### CROSS REFERENCE TO RELATED APPLICATIONS

This application claims the benefit of U.S. Provisional Patent Application No. 61/497,332, filed Jun. 15, 2011, which is incorporated by reference herein, in its entirety.

### FIELD OF THE INVENTION

The present invention relates generally to cardiac treatment. More particularly, the present invention relates to a device for cardiac ablation.

### BACKGROUND

The term cardiac arrhythmia refers to a large group of conditions in which there is abnormal electrical activity in the heart. Catheter ablation is a medical procedure that can be used to treat some types of cardiac arrhythmias. During catheter ablation, a long, thin, flexible tube that has an ablation electrode on the end is directed through a blood vessel into the patient's heart. Certain portions of heart tissue are then destroyed by the application of electrical energy through the ablation electrode.

One common procedure relates to atrial fibrillation in which tissue within a pulmonary vein causes arrhythmias in an atrium of the heart. For such a procedure, it is necessary to ablate tissue around the entire edge of the pulmonary vein to isolate the electrical interference from the tissue in the pulmonary vein. For this procedure to be successful, complete electrical isolation is necessary. If a spot is missed, the procedure will not be successful. In addition, too much ablation can cause damage to the heart which presents a danger for the patient.

Currently, surgeons perform the ablation procedure free-hand, without a useful mechanism with which to assist guiding the ablation to the desired path and for the desired depth. There thus remains a need for improved ablation catheters.

### SUMMARY OF THE INVENTION

In accordance with an aspect of the present invention, an ablation catheter, includes a catheter body having a distal end and a proximal end. An ablation electrode is attached to the distal end of the catheter body, and a pivot needle is arranged to extend from said catheter body while in use. The pivot needle provides support to assist with directing said ablation electrode along a selected path in contact with tissue during ablation.

In accordance with another aspect of the present invention, the pivot needle includes a gripping structure at a distal tip to facilitate attachment to tissue. The gripping structure is a barb structure, and the barb structure is formed from a soft material to facilitate removal from tissue after being attached. The soft material is a polymer material. The pivot needle further includes a depth guard to help prevent the insertion of said pivot needle into tissue beyond a predetermined depth, and said pivot needle is extendable from and retractable to said catheter body.

### BRIEF DESCRIPTION OF THE DRAWINGS

FIG. 1A illustrates a distal end of an ablation catheter according to an embodiment of the present invention.

FIG. 1B illustrates an exemplary embodiment of a barb structure at a distal tip of a pivot needle according to an embodiment of the present invention.

FIG. 1C illustrates an exemplary embodiment of a depth guard according to an embodiment of the current invention.

FIG. 1D illustrates an exemplary embodiment of a pivot needle in conjunction with a distal end of an ablation catheter according to an embodiment of the present invention.

FIG. 2 illustrates a schematic showing how an ablation catheter according to an embodiment of the current invention can be used to assist in ablating a closed loop around a pulmonary vein.

FIG. 3 illustrates a pivot structure fixed within a pulmonary vein according to an embodiment of the invention.

FIG. 4 illustrates an ablation catheter according to an embodiment of the present invention.

### DETAILED DESCRIPTION

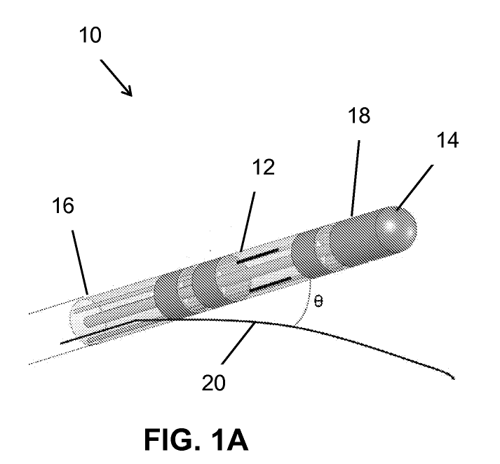

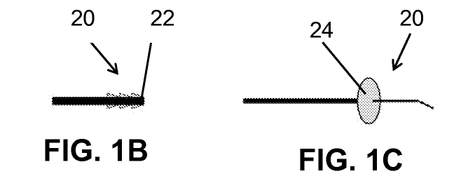

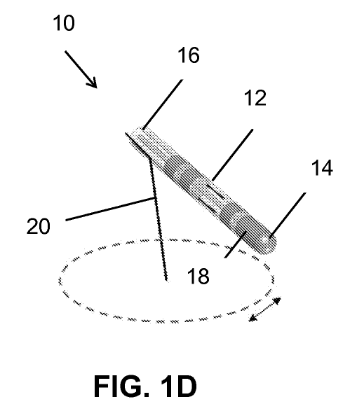

FIG. 1A is an illustration of a distal end of an ablation catheter according to an embodiment of the current invention. The ablation catheter 10 includes a catheter body 12 that has a distal end 14 and a proximal end 16, an ablation electrode 18 attached to the distal end 14 of the catheter body 12, and a pivot needle 20 arranged to extend from the catheter body while the ablation catheter is in use. The pivot needle 20, illustrated in FIG. 1A, provides support to assist with directing the ablation electrode 18 along a selected path in contact with tissue during ablation. In an embodiment of the current invention, the pivot needle 20 can have a gripping structure, such as barbs, hooks or clamps, for example, at a distal tip to facilitate attachment to tissue.

FIG. 1B illustrates an example of a barb structure 22 at the distal tip of the pivot needle 20. The barb structure 22 can be formed from a soft material to facilitate removal from tissue after being attached according to some embodiments of the current invention. In some embodiments, the soft material can be a polymer material, for example.

In some embodiments, the pivot needle 20 can further include a depth guard 24 to help prevent the insertion of the pivot needle 20 into tissue beyond a predetermined depth. FIG. 1C illustrates an example of a depth guard 24 according to an embodiment of the current invention. In some embodiments, the pivot needle 20 can be extendable from and retractable to the catheter body 12. For example, the pivot needle 20 can be a long wire that extends along the length of the ablation catheter. The catheter body 12 can define a lumen through which wires connected to the ablation electrode and other sensors can be contained. Similarly, the pivot needle 20 can be contained within the lumen according to some embodiments of the current invention. The pivot needle 20 can be partially, or totally, electrically insulated according to some embodiments. Alternatively, the catheter body 12 can define a double, or multiple, lumens such that the pivot extends along one lumen. A grasping and/or locking mechanism can be included at the proximal end 16 of the ablation catheter 10 such that the pivot needle 20 can be extended and contracted and held at a fixed amount of extension, or held retracted, while in use. The pivot needle 20 can also be pre-bent at a selected position and angle, such as θ, as illustrated in FIG. 1A, according to the particular application. In some embodiments, the pivot needle 20 could be constructed from a shape memory alloy to provide a further degree of control over the shape of the extended pivot needle, for example.

In operation, the ablation catheter 10 is maneuvered through blood vessels into the patient's heart while the pivot needle 20 is in a retracted configuration. Once the distal end 14 of the ablation catheter 10 is close to the target tissue within the heart, the pivot needle 20 is extended. In some embodiments, the pivot needle 20 can have a pre-formed bend such that it springs out to an angle such as is depicted in FIGS. 1A and 1D, for example.

However, the general concepts of the current invention are not limited to this particular embodiment. In other embodiments, the pivot needle 20 can extend substantially straight out while the end of the ablation catheter is curved. One can see from FIG. 1D, that the pivot needle 20 can help the user ablate along an accurate circle about an attachment point. In other embodiments, the pivot needle 20 may be used in an unattached configuration in which it serves as a reference or can rest against or alongside a structure.

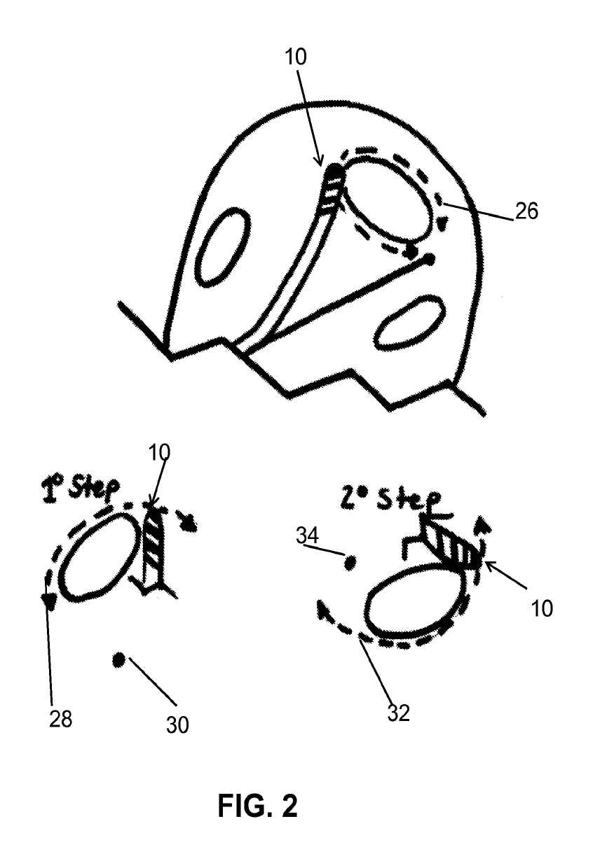

FIG. 2 is a schematic illustration to show how an ablation catheter 10 according to an embodiment of the current invention can be used to assist in ablating a closed loop 26 around a pulmonary vein. In the example of FIG. 2, a first arc 28 of ablation is performed with the pivot needle fixed in a first point 30 and then a second arc 32 of ablation that intersects the first arc 28 at two portions is performed with the pivot needle fixed at a second point 34. In general, any number of fixed points can be used, as desired. However, one can see from this example, that two fixed points 30, 34 may often be sufficient to perform the isolation of the pulmonary vein.

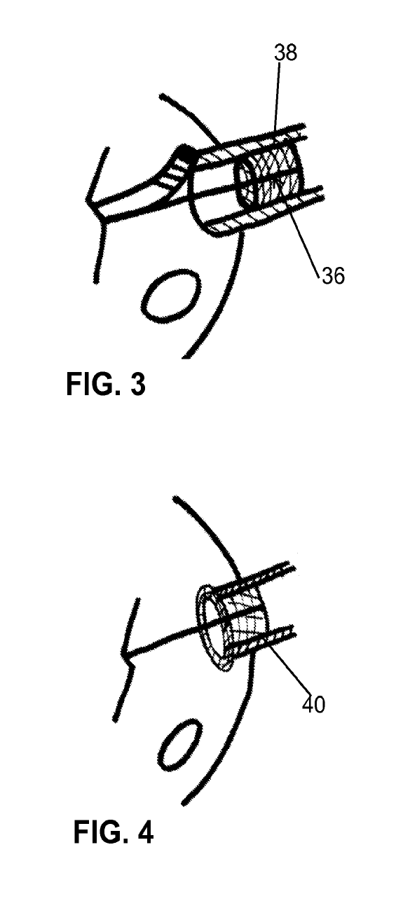

Alternative embodiments can include pivot needles and/or other structures to similarly serve as a guide. For example, FIG. 3 is an example of an embodiment in which a pivot structure 36 fixes within the pulmonary vein, for example. In this example, instead of a pivot needle, a wire has an expandable and contractible mesh structure 38 that expands to attach within the pulmonary vein, for example, and contracts for removal.

FIG. 4 is a schematic illustration of another embodiment of an ablation catheter according to the current invention. This embodiment is similar to the embodiment of FIG. 3 in that it has an expandable and contractible mesh structure 40. However, the mesh structure also is electrically connected to provide the ablation electrode structures. In this embodiment, the mesh structure is electrically insulating except at the edge, which can be aligned with the edge of the pulmonary vein, for example.

Concepts of the current invention are described by way of examples herein. The broad concepts of the current invention are not limited to only the specific embodiments described. One of ordinary skill in this art should recognize, in view of the teachings herein, that many modifications are possible without departing from the general concepts of this invention.

1. An ablation catheter, comprising:
a catheter body having a distal end and a proximal end;
an ablation electrode attached to said distal end of said catheter body; and
a pivot needle arranged to extend from said catheter body while in use,
wherein said pivot needle provides support to assist with directing said ablation electrode along a selected path in contact with tissue during ablation.

2. The ablation catheter according to claim 1, wherein said pivot needle comprises a gripping structure at a distal tip to facilitate attachment to tissue.

3. The ablation catheter according to claim 2, wherein said gripping structure is a barb structure.

4. The ablation catheter according to claim 3, wherein said barb structure is formed from a soft material to facilitate removal from tissue after being attached.

5. The ablation catheter according to claim 4, wherein said soft material is a polymer material.

6. The ablation catheter according to claim 1, wherein said pivot needle further comprises a depth guard to help prevent the insertion of said pivot needle into tissue beyond a predetermined depth.

7. The ablation catheter according to claim 1, wherein said pivot needle is extendable from and retractable to said catheter body.
